(19) United States
(12) Patent Application Publication
(10) Pub. No.: US 2023/0355283 A1
(43) Pub. Date: Nov. 9, 2023

(54) DEFORMABLE METAL PLATE FOR USE IN FRACTURE TREATMENT

(71) Applicant: Myung Sik PARK, Jeonju-si (KR)

(72) Inventor: Myung Sik PARK, Jeonju-si (KR)

(21) Appl. No.: 18/142,911

(22) Filed: May 3, 2023

(30) Foreign Application Priority Data
May 6, 2022 (KR) 10-2022-0056197

(51) Int. Cl. A61B 17/80

(52) U.S. Cl. CPC A61B 17/8085; A61B 17/8004; A61B 17/8023

(57) ABSTRACT

Provided is a deformable metal plate for use in fracture treatment, in which the length and angle of a single metal plate for use in fracture treatment are variable and the width thereof can also be adjusted when the length and angle are varied. The deformable metal plate for use in fracture treatment includes a metal plate body having body screw insertion holes at predetermined intervals, a plurality of uneven portions disposed on at least one side of the metal plate body along a longitudinal direction of the metal plate body at predetermined intervals, and a variable metal plate configured to be slid along and coupled to a part of the plurality of uneven portions, the variable metal plate having, an uneven coupling portion, and an extension arm extending from the sliding and coupling body. A variable screw insertion hole is defined at an end of the extension arm and when the uneven coupling portion is fixed onto the metal plate body, the extension arm has a predetermined angle with the longitudinal direction of the metal plate body.

### CROSS-REFERENCE TO RELATED APPLICATION

This application claims priority to and the benefit of Korean Patent Application No. 10-2022-0056197 filed in the Korean Intellectual Property Office on May 6, 2022, the entire contents of which are incorporated herein by reference.

### BACKGROUND

### 1. Field

The present disclosure relates to a deformable metal plate for use in fracture treatment, and more particularly, to a deformable metal plate for use in fracture treatment, in which the length and angle of a single metal plate for use in fracture treatment are variable and the width thereof can also be adjusted when the length and angle are varied.

### 2. Description of the Related Art

In general, a metal plate is used for fracture treatment as a means for fixing bones to each other. As an example, Korean Patent Registration No. 10-0423699, entitled “Metal Plate for Fracture Treatment”, or Korean Utility Model Registration No. 20-0309570, entitled “Intramedullary Metal Plate for Fracture Treatment”, is disclosed. They are all a single metal plate configured to fix bones to each other.

However, only the single metal plate cannot flexibly cope with various fracture angles and gaps between bones. For this reason, a solution to this problem is required.

### SUMMARY OF THE INVENTION

The present disclosure has been proposed to solve the above problems and aims to provide a deformable metal plate for use in fracture treatment, in which the length and angle of a single metal plate for use in fracture treatment are variable and the width thereof can also be adjusted when the length and angle are varied.

In order to solve the above problems, a deformable metal plate for use in fracture treatment according to an embodiment of the present disclosure includes: a metal plate body provided with screw insertion holes at predetermined intervals; a plurality of uneven portions provided at predetermined intervals along a length of the metal plate body; and a variable metal plate selectively coupled to some of the plurality of uneven portions and including a screw insertion hole at an end thereof, wherein a position of the screw insertion hole is variable according to a screw insertion position by adjusting a coupling position of the variable metal plate to the metal plate body.

Here, the variable metal plate may be a horizontal variable metal plate that has an uneven coupling portion coupled to the uneven portion at one end, has a length parallel to a length of the metal plate body at the uneven coupling portion, and includes the screw insertion hole at the other end, a vertical variable metal plate that has an uneven coupling portion coupled to the uneven portion at one end, has a length perpendicular to the length of the metal plate body at the uneven coupling portion, and includes the screw insertion hole at the other end, and an inclined variable metal plate that has an uneven coupling portion coupled to the uneven portion at one end, having an inclination with an angle with respect to the length of the metal plate body at the uneven coupling portion, and includes the screw insertion hole at the other end.

In addition, the variable metal plate may form an auxiliary metal plate body having the same shape as the metal plate body, except for the length, at the end where the screw insertion hole is formed.

In addition, the variable metal plate may form a screw penetration hole in the uneven coupling portion, and the uneven portion may be provided at a position corresponding to the position of the screw insertion hole of the metal plate body. Therefore, when the variable metal plate is coupled to the uneven portion through the uneven coupling portion, the screw penetration hole and the screw insertion hole may be formed to face each other.

In addition, the uneven portion may not be provided at a position corresponding to the position of the screw insertion hole of the metal plate body.

In addition, the metal plate body may form a concave portion between the plurality of uneven portions.

In addition, the uneven portion may form one or more uneven protrusions, and the uneven coupling portion may form a cutout portion in which a plurality of cutout grooves are densely formed to correspond to all or part of the uneven protrusions. After the cutout groove is caught on the uneven protrusion, the uneven coupling portion is compressed with a compression fixer so that the variable metal plate is fixed to the metal plate body.

In addition, the uneven protrusions may be provided one by one on at least both sides of one uneven portion. The gap between the uneven protrusions on both sides in one uneven portion may be equal to the gap between the uneven protrusions of the uneven portions adjacent to each other.

### BRIEF DESCRIPTION OF THE DRAWINGS

FIG. 1 illustrates one mode of a deformable metal plate for use in fracture treatment according to an embodiment of the present disclosure.

FIG. 2 illustrates another mode of a deformable metal plate for use in fracture treatment according to another embodiment of the present disclosure.

FIG. 3 is a detailed diagram of a deformable metal plate, which is an element of the deformable metal plate for use in fracture treatment according to an embodiment of the present disclosure.

FIG. 4 is a detailed diagram of a horizontal deformable metal plate, which is an element of the deformable metal plate for use in fracture treatment according to an embodiment of the present disclosure.

FIG. 5 is a detailed diagram of a vertical deformable metal plate, which is an element of the deformable metal plate for use in fracture treatment according to an embodiment of the present disclosure.

FIG. 6 is a detailed diagram of an inclined deformable metal plate, which is an element of the deformable metal plate for use in fracture treatment according to an embodiment of the present disclosure.

FIG. 7 is a side exemplary view of a variable metal plate according to an embodiment of the present invention surrounding a body part.

FIG. 8 is a detailed diagram of a bone fixing screw, which is an element of the deformable metal plate for use in fracture treatment according to an embodiment of the present disclosure.

FIGS. 9A and 9B are diagrams illustrating examples of using the deformable metal plate for use in fracture treatment according to an embodiment of the present disclosure.

FIG. 10 is a diagram illustrating a deformable metal plate with an auxiliary metal plate body according to an embodiment of the present disclosure.

FIG. 11 is a diagram illustrating an example of using the deformable metal plate with the auxiliary metal plate body of FIG. 10.

### DETAILED DESCRIPTION OF THE EMBODIMENTS

Hereinafter, the description of the present disclosure with reference to the drawings is not limited to specific embodiments, and various modifications may be made thereto and various embodiments may be provided. In addition, the following description should be understood to include all changes, equivalents, or substitutes included in the spirit and scope of the present disclosure.

In the following description, the terms such as “first” and “second” are terms used to describe various elements, are not limited in meaning itself, and are only used to distinguish one element from another.

Like reference numbers used throughout the present specification represent like elements.

The singular forms as used herein are intended to include the plural forms as well unless the context clearly indicates otherwise. The terms “comprise,” “include,” and “have” as used herein are inclusive and therefore specify the presence of stated features, integers, steps, operations, elements, components, or combinations thereof, but do not preclude the presence or addition of one or more other features, integers, steps, operations, elements, components, or combinations thereof.

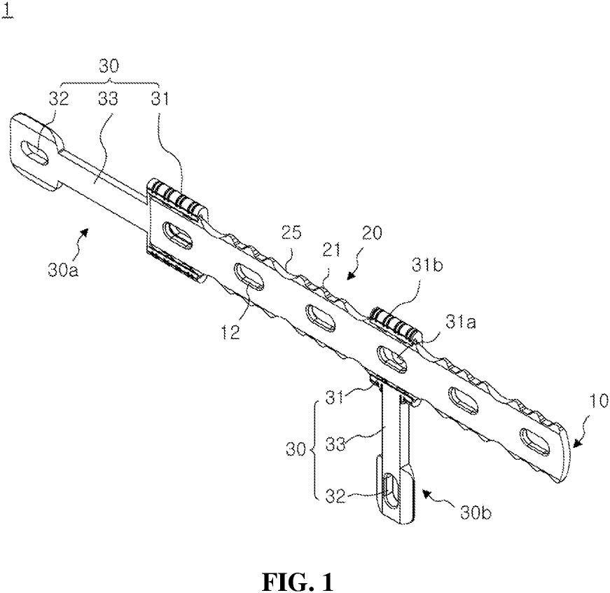

Hereinafter, an embodiment of the present disclosure will be described in detail with reference to FIGS. 1 to 11 attached herein.

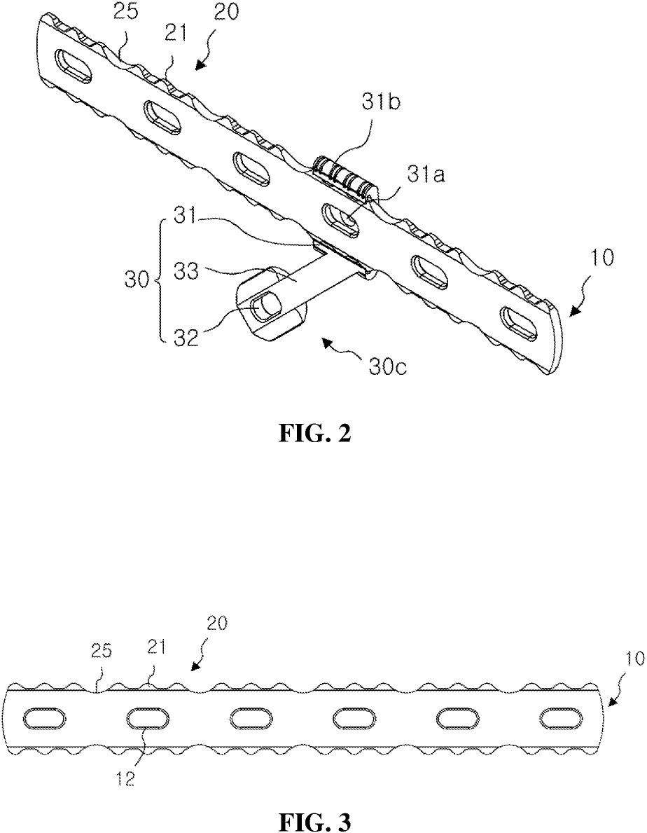

FIG. 1 illustrates one mode of a deformable metal plate for use in fracture treatment according to an embodiment of the present disclosure, FIG. 2 illustrates another mode of a deformable metal plate for use in fracture treatment according to another embodiment of the present disclosure, and FIG. 3 is a detailed diagram of a deformable metal plate, which is an element of the deformable metal plate for use in fracture treatment according to an embodiment of the present disclosure.

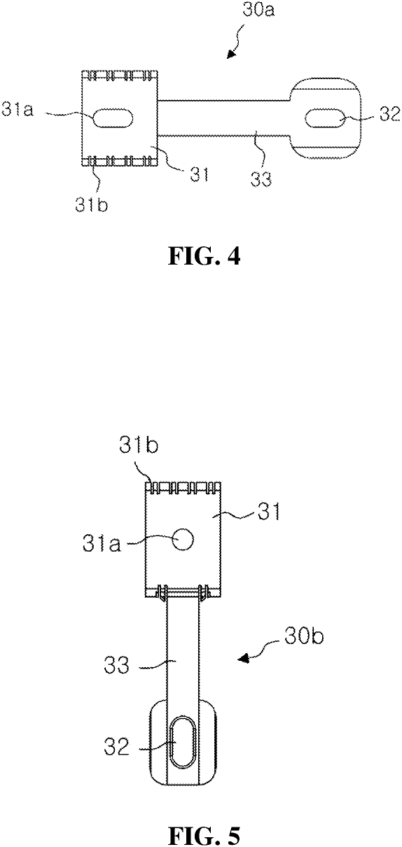

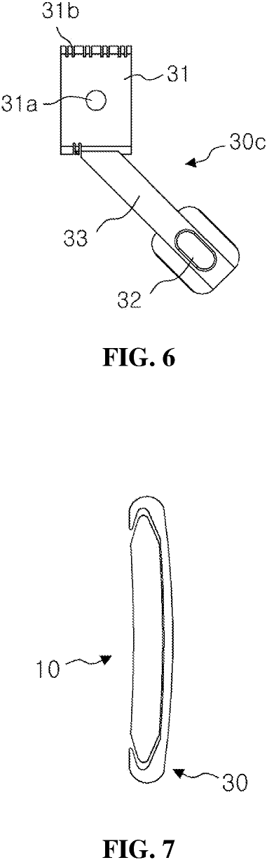

In addition, FIG. 4 is a detailed diagram of a horizontal deformable metal plate, which is an element of the deformable metal plate for use in fracture treatment according to an embodiment of the present disclosure, FIG. 5 is a detailed diagram of a vertical deformable metal plate, which is an element of the deformable metal plate for use in fracture treatment according to an embodiment of the present disclosure, and FIG. 6 is a detailed diagram of an inclined deformable metal plate, which is an element of the deformable metal plate for use in fracture treatment according to an embodiment of the present disclosure.

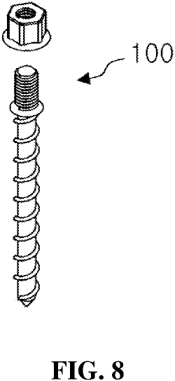

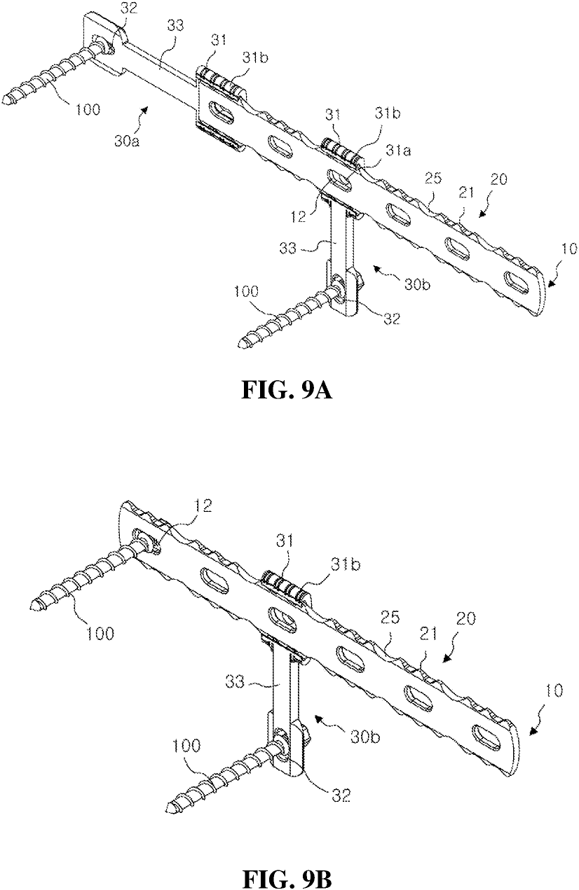

In addition, FIG. 7 is a side exemplary view of a variable metal plate according to an embodiment of the present invention surrounding a body part, FIG. 8 is a detailed diagram of a bone fixing screw, which is an element of the deformable metal plate for use in fracture treatment according to an embodiment of the present disclosure, and FIGS. 9A and 9B are diagrams illustrating examples of using the deformable metal plate for use in fracture treatment according to an embodiment of the present disclosure.

Referring to FIGS. 1 to 9B, the deformable metal plate 1 for use in fracture treatment according to an embodiment of the present disclosure may include a metal plate body 10, an uneven portion 20, and a variable metal plate 30.

Specifically, the metal plate body 10 may have screw insertion holes 12 provided at predetermined intervals, and a plurality of uneven portions 20 may be provided at predetermined intervals along the length of the metal plate body 10. In addition, the uneven portion 20 may be provided to be symmetrical to the upper and lower sides with respect to the metal plate body 10. That is, the metal plate body 10 may be provided with a plurality of screw insertion holes 12 and a plurality of uneven portions 20 along the length. At this time, the uneven portion 20 may be provided along the upper and lower lengths of the metal plate body 10.

At this time, although the drawings illustrate that the screw insertion hole 12 and the uneven portion 20 are provided at positions corresponding to each other, this is only a preferred form and the present disclosure is not necessarily limited thereto. The screw insertion hole 12 and the uneven portion 20 may be provided at positions that do not correspond to each other. This will be described in detail below.

The variable metal plate 30 is a means coupled to the metal plate body 10 and may be selectively coupled to some of the plurality of uneven portions 20. That is, the variable metal plate 30 may be selectively coupled to one of the plurality of uneven portions 20 or may be selectively coupled to two or more uneven portions 20. In this case, the variable metal plate 30 may be coupled to the plurality of uneven portions 20 while changing the position thereof. By adjusting the coupling position of the variable metal plate 30 with respect to the metal plate body 10, the present disclosure can achieve the effect of the deformable metal plate 1.

More specifically, first, by providing the plurality of screw insertion holes 12 in the metal plate body 10, at least two bone fixing screws 100 may be inserted at different positions. In this manner, the metal plate body 10 may be placed between the bone and the fractured bone, and the bone fixing screws 100 may be respectively inserted into at least two screw insertion holes 12 matched to the gap between the bones and may be respectively fixed to the bones. Therefore, the metal plate body 10 may be fitted and fixed between the bone and the fractured bone.

However, only the single metal plate body 10 cannot flexibly cope with various fracture angles and gaps between bones. For this reason, the present disclosure may be flexibly applied to more diverse fracture types by varying the length or angle of the metal plate body 10 and the stride for the length or angle through the variable metal plate 30.

To this end, the variable metal plate 30 may have an uneven coupling portion 31 and an extension arm 33. One end of the variable metal plate 30 has the uneven coupling portion 31 and another end of the variable metal plate 30 has a screw insertion hole 32. Here, The uneven coupling portion 31 is designed to slide and couple onto the metal plate body 10, and the screw insertion hole 32 allows for screw insertion. An extension arm 33 extends from the uneven coupling portion 31, connecting it to the screw insertion hole 32. The extension arm 33 rotates around a single point of the uneven coupling portion 31, forming a predetermined angle with the longitudinal direction of the metal plate body 10. When the uneven coupling portion 31 is fixed onto the metal plate body 10, the extension arm 33 has a predetermined angle with the longitudinal direction of the metal plate body 10.

In addition, the variable metal plate 30 may include a horizontal variable metal plate 30a for varying the length and stride of the metal plate body 10, and a vertical variable metal plate 30b and an inclined variable metal plate 30c for varying the angle and stride of the metal plate body 10.

Specifically, the horizontal variable metal plate 30a is a metal plate having a length parallel to the length of the metal plate body 10 when coupled to the metal plate body 10 through the uneven coupling portion 31, the vertical variable metal plate 30b is a metal plate having a length perpendicular to the length of the metal plate body 10 when coupled to the metal plate body 10 through the uneven coupling portion 31, and the inclined variable metal plate 30c is a metal plate having an angle with the metal plate body 10 when coupled to the metal plate body 10.

The horizontal variable metal plate 30a, the vertical variable metal plate 30b, and the inclined variable metal plate 30c may each have a predetermined length from the uneven coupling portion 31 of the same shape, and the lengths thereof may be formed to extend in different directions from the uneven coupling portion 31. Therefore, among the horizontal variable metal plate 30a, the vertical variable metal plate 30b, and the inclined variable metal plate 30c, one may have a length parallel to the metal plate body 10, another may have a length perpendicular to the metal plate body 10, and the other may have a length in an oblique direction.

The screw insertion holes 32 are provided at the ends of the horizontal variable metal plate 30a, the vertical variable metal plate 30b, and the inclined variable metal plate 30c, so that the bone fixing screws 100 can be inserted thereinto in the same way as the metal plate body 10. One bone fixing screw 100 may be inserted into the metal plate body 10, and another bone fixing screw may be inserted into the variable metal plate 30.

In this case, when the horizontal variable metal plate 30a is coupled to the metal plate body 10 as illustrated in the drawings, the length of the metal plate body 10 may be varied so that the bone fixing screws 100 can be fixed at wider intervals in the horizontal direction. When the vertical variable metal plate 30b or the inclined variable metal plate 30c is coupled to the metal plate body 10, the angle of the metal plate body 10 may be varied so that the bone fixing screw 100 can be fixed in the angular direction.

On the other hand, the screw insertion hole 12 of the metal plate body 10 and the screw insertion holes 32 of the variable metal plates 30a, 30b, and 30c may each be formed in the form of an elongated hole that is elongated in both directions, so that the insertion of the bone fixing screw 100 or the position adjustment of the metal plate body 10 can be more flexible.

In addition, as described above, the screw insertion hole 12 of the metal plate body 10 and the uneven portion 20 may be provided at positions corresponding to each other as illustrated in the drawings. Although not illustrated in the drawings, the screw insertion hole 12 of the metal plate body 10 and the uneven portion 20 may be provided at positions that do not correspond to each other.

When the screw insertion hole 12 of the metal plate body 10 and the uneven portion 20 are provided at positions that do not correspond to each other, there is an advantage in that the screw insertion hole 12 of the metal plate body 10 can be used to avoid interference of the variable metal plate 30 coupled to the metal plate body 10.

On the other hand, when the screw insertion hole 12 and the uneven portion 20 of the metal plate body 10 are provided at positions corresponding to each other, a screw penetration hole 31a may be formed in the uneven coupling portion 31 of the variable metal plate 30. When the variable metal plate 30 is coupled to the uneven portion 20 through the uneven coupling portion 31, the screw penetration hole 31a and the screw insertion hole 12 of the metal plate body 10 may be formed to face each other. In this way, when the screw penetration hole 31a and the screw insertion hole 12 face each other, the screw penetration hole 31a and the screw insertion hole 12 may be screwed to each other, thereby more firmly fixing the variable metal plate 30 to the metal plate body 10.

As described above, since the horizontal or vertical variable metal plate 30a or 30b is selectively coupled to the uneven portion 20 formed at a position corresponding to or not corresponding to the screw insertion hole 12 of the metal plate body 10, the above-described effects may be exhibited and it is possible to flexibly cope with various fracture angles or gaps between bones.

On the other hand, the metal plate body 10 may form concave portions 25 between the plurality of uneven portions 20. Here, the concave portion 25 is a means for facilitating a user’s gripping while reducing the weight of the metal plate body 10, easily distinguishing the length of the metal plate body 10 with the naked eye, and in particular, facilitating deformation such as bending of the metal plate. The concave portion 25 may have a shape that is deeply recessed toward the metal plate body 10 rather than the uneven portion 20. The concave portion 25 may have a “v” shape or an arch shape, as illustrated in the drawings. However, the present disclosure is not necessarily limited thereto, and the concave portion 25 may have various shapes.

In addition, the uneven portion 20 may form one or more uneven protrusions 21. When the uneven portion 20 forms the uneven protrusion 21, the uneven coupling portion 31 may form a cutout groove portion 31b in which a plurality of cutout grooves (not illustrated) are densely formed to correspond to all or part of the uneven protrusion 21. In this case, the cutout groove 31b is formed so as to be caught on the uneven protrusion 21. When the variable metal plate 30 is coupled to the metal plate body 10, the cutout groove 31b may be caught on the uneven protrusion 21, and the uneven coupling portion 31 may be compressed with a compression fixer (not illustrated) to have a strong fixing force.

To this end, it is manufactured so that a slight gap is formed between the cutout groove 31b and the uneven protrusion 21 to the extent of being lightly caught, making it easy to correct the position before pressing with a compression fixer. When the compression is performed with the compression fixer, the uneven protrusion 21 may engage with the cutout groove 31b to have a more robust fixing force. At this time, since a plurality of cutout grooves forming the cutout groove 31b and substantially engaging the uneven protrusion 21 are provided, there is also an advantage in that the uneven protrusion 21 can be easily caught without special efforts to match the position of the uneven protrusion 21.

On the other hand, the uneven protrusion 21 preferably has a sharp end so that the cutout groove 31b can be easily caught, but the present disclosure is not limited thereto and the uneven protrusion 21 may have a blunt end.

In addition, the uneven protrusions 21 may be provided one by one on at least both sides of one uneven portion 20. The gap between the uneven protrusions 21 on both sides in one uneven portion 20 may be formed to be equal to the gap between one of the uneven protrusions 21 on both sides and the facing uneven protrusion 21 of the other adjacent uneven portions 20. Therefore, the positions to which the variable metal plate 30 is fixed may be more diversified, and the length or angle adjustment may be more easily performed.

The plurality of uneven portions 20 may be disposed on both sides of the metal plate body 10 along the longitudinal direction of the metal plate body 10 at predetermined intervals.

Referring to FIG. 9A, a plurality of the variable metal plates 30 can be attached to the metal plate body 10. Each extension arm of the plurality of the variable metal plates 30 may have a different angle with the longitudinal direction of the metal plate body 10 from one another.

In addition, in the horizontal variable metal plate 30a, the vertical variable metal plate 30b, and the inclined variable metal plate 30c, an auxiliary metal plate body 40 having the same shape as the metal plate body 10 may be formed at the end where the screw insertion hole 32 is formed.

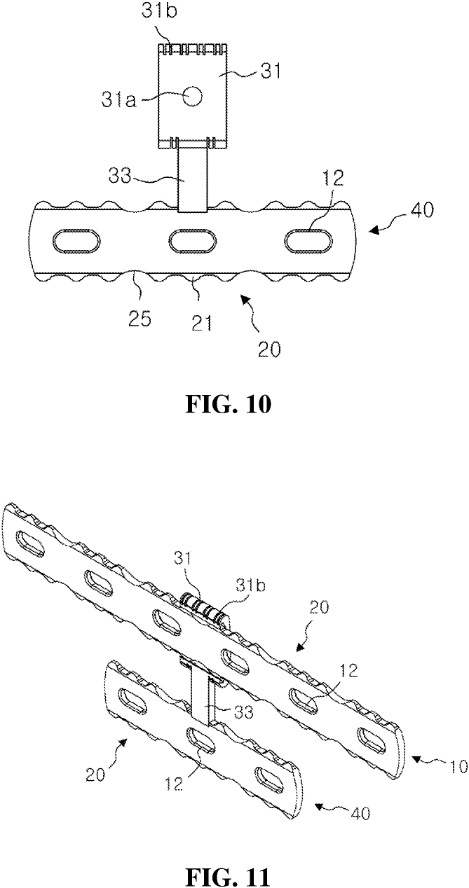

FIG. 10 is a diagram illustrating a deformable metal plate with an auxiliary metal plate body according to an embodiment of the present disclosure, and FIG. 11 is a diagram illustrating an example of using the deformable metal plate with the auxiliary metal plate body of FIG. 10.

Referring to FIGS. 10 and 11, the auxiliary metal plate body 40 may have the same shape as the metal plate body 10. The uneven portion 20 and the screw insertion hole 12 may be formed in the same manner as described in the metal plate body 10. The auxiliary metal plate body 40 may be symmetrical with the same length as the metal plate body 10 or may be different only in length.

When the auxiliary metal plate body 40 is used for the vertical variable metal plate 30b, two parallel dual metal plate bodies 10 may be implemented. When the auxiliary metal plate body 40 is used for the horizontal variable metal plate 30a, the metal plate body 10 may be formed to extend in the vertical direction. In other words, the auxiliary metal plate body 40 may have various predetermined angles with the extension arm 33.

As described above, since the auxiliary metal plate body 40 may have the same shape as the metal plate body 10 while being different from the metal plate body 10 only in length according to the setting, a detailed description thereof is omitted.

A deformable metal plate for use in fracture treatment according to an embodiment of the present disclosure may vary the length and angle of a single metal plate for use in fracture treatment and may also adjust the width thereof when the length and angle are varied, so that the deformable metal plate can be applied flexibly according to more various fracture types.

In addition, the deformable metal plate for use in fracture treatment according to an embodiment of the present disclosure has a simple structure, easy use, and low manufacturing or maintenance costs.

On the other hand, the deformable metal plate for use in fracture treatment according to an embodiment of the present disclosure is not limited to the effects described above, and may further include all effects predictable from the specification and drawings.

Although the embodiments of the present disclosure have been described with reference to the accompanying drawings, those of ordinary skill in the art to which the present disclosure pertains will understand that the present disclosure may be embodied in other specific forms without changing the technical spirit or essential features. Therefore, it will be understood that the embodiments described above are illustrative in all aspects and are not restrictive.

Description of symbols

| | | | |
|---|---|---|---|
| 1: | deformable metal plate | 10: | metal plate body |
| 12: | screw insertion hole | 20: | uneven portion |
| 21: | uneven protrusion | 25: | concave portion |
| 30: | variable metal plate | 30a: | horizontal variable metal plate |
| 30b: | vertical variable metal plate | 30c: | inclined variable metal plate |
| 31: | uneven coupling portion | 31a: | screw penetration hole |
| 31b: | cutout groove | 32: | screw insertion hole |
| 33: | extension arm | 40: | auxiliary metal plate body |
| 100: | bone fixing screw | | |

What is claimed is:

1. A deformable metal plate for bone fracture treatment, the deformable metal plate comprising:
a metal plate body having body screw insertion holes at predetermined intervals;
a plurality of uneven portions disposed on at least one side of the metal plate body along a longitudinal direction of the metal plate body at predetermined intervals; and
a variable metal plate configured to be slid along and coupled to a part of the plurality of uneven portions, the variable metal plate having:
an uneven coupling portion, and
an extension arm extending from the uneven coupling portion, wherein a variable screw insertion hole is defined at an end of the extension arm,
wherein when the uneven coupling portion is fixed onto the metal plate body, the extension arm has a predetermined angle with the longitudinal direction of the metal plate body.

2. The deformable metal plate of claim 1, wherein when the uneven coupling portion is fixed onto the metal plate body, the extension arm is in a parallel direction with the longitudinal direction of the metal plate body, and the variable screw insertion hole is positioned along an extended line of a length of the metal body plate.

3. The deformable metal plate of claim 1, wherein when the uneven coupling portion is fixed onto the metal plate body, the extension arm is normal to the longitudinal direction of the metal plate body.

4. The deformable metal plate of claim 1, wherein when the uneven coupling portion is fixed onto the metal plate body, the extension arm has an angle between 0 and 90 degrees with the longitudinal direction of the metal plate body.

5. The deformable metal plate of claim 1, wherein the variable metal plate further comprises an auxiliary metal plate body attached to the end of the extension arm and having a plurality of auxiliary screw insertion holes.

6. The deformable metal plate of claim 5, wherein the auxiliary metal plate body has a different length from the metal plate body.

7. The deformable metal plate of claim 5, wherein the auxiliary metal plate body has a predetermined angle with the extension arm.

8. The deformable metal plate of claim 1, wherein the plurality of uneven portions disposed on both sides of the metal plate body along the longitudinal direction of the metal plate body at predetermined intervals.

9. The deformable metal plate of claim 1, wherein a plurality of variable metal plates are attached to the metal plate body.

10. The deformable metal plate of claim 9, wherein each extension arm of the plurality of the variable metal plates has a different angle with the longitudinal direction of the metal plate body from one another.
